(19) United States
(12) Patent Application Publication
(10) Pub. No.: US 2005/0244957 A1
(43) Pub. Date: Nov. 3, 2005

(54) Regenerating tank

(75) Inventor: Raymond W. Stock, South Jordan, UT (US)

Correspondence Address: Starkweather and Associates;Suite 200, 9035 South 1300 East, Sandy, UT, 84094, US

(73) Assignee: HEALTHY SOILS, INC.

(21) Appl. No.: 11/119,124

(22) Filed: Apr. 28, 2005

Related U.S. Application Data
(60) Provisional application No. 60/566,893, filed on Apr. 29, 2004.

(51) Int. Cl. C12M 1/06

(52) U.S. Cl. 435/289.1; 435/295.1; 435/286.5

(57) ABSTRACT

A microorganism regeneration system, method and apparatus for providing a regenerating supply of microorganisms. There is a hollow vessel, a circulation system, an inlet system, an outlet system, a microorganism quantity, a nutrient quantity, and/or a water quantity. The hollow vessel contains liquids; the circulation system disposed within the hollow vessel is configured to circulate and aerate liquid; the inlet system coupled to the hollow vessel is configured to cause entry of an aqueous liquid into the hollow vessel only when a liquid volume is below a determined threshold; the outlet system coupled to the hollow vessel is configured to controllably permit discharge of liquid from the hollow vessel; the microorganism quantity has at least a first generation of microorganisms; the nutrient quantity is in an amount sufficient to sustain reproduction of the microorganism quantity for a reproduction period; the water quantity is in an amount sufficient to cause the liquid volume to be at the determined threshold.

### BACKGROUND OF THE INVENTION

This invention claims priority, under 35 U.S.C. § 119, to the U.S. Provisional Patent Application No. 60/566,893 to Raymond Stock filed on Apr. 29, 2004, which is incorporated by reference herein.

### FIELD OF THE INVENTION

The present invention relates to biological reproduction devices and systems, specifically biological regeneration methods, devices, and systems such as microorganism reproduction tanks.

### DESCRIPTION OF THE RELATED ART

Biologicals, such as some kinds of bacteria, are a vital part of any renewable ecosystem. In particular, biologicals permit repeated extraction of resources from ecosystems, including systems such as farms and ranches of the agricultural industry, by enhancing the cyclical nature of the ecosysterns. Without healthy biological components, these ecosystems operate at reduced efficiency and may require substantial increases in added resources. Further, the added resources may fail to compensate for the missing biologicals, as some functions provided by the biologicals are extremely difficult and/or expensive to mimic using other means.

The agriculture industry is an important component of society because it is responsible for producing food for human and animal consumption. In furtherance of this responsibility, the agricultural industry relies heavily on the soil from which crops are grown and cultivated. Over time, the repeated use of soil for growing crops can result in a diminishment of the available nutrients. As this occurs, plants can begin to show signs of inadequate nutrition by having stunted growth and/or poor health characteristics. To overcome the problems of nutrient depleted soil, farmers have been adding biological and/or nutrient containing substances to their soil for years, of which the most common is the application of animal manure. Additionally, various fertilizers have been developed for the purpose of stimulating the biological activity and nutrient level of farming soil.

While manure and fertilizers have been developed and used for many years, the results have been less than optimal. This is because some of the nutrient containing substances may be required to be applied several times a year, and some require applications for at least two or three years before any cost effective results are observed. Additionally, some substances are difficult to apply because of their nature to clump or otherwise not be spread evenly across the soil. Accordingly, this can cause the soil to be over-fertilized in some areas and under-fertilized in others, which results in overall poor growing conditions.

Over the past decades there has been a shift from smaller localized family farms toward larger integrated confinement agricultural operations. Specifically, large agricultural operations referred to as concentrated animal feeding operations (CAFO's) may utilize confinement barns to house a large number of livestock such as swine, poultry or dairy cows. Using the swine industry as an example, often numerous hog-confinement operations are grouped in close proximity forming “mega-farms” which may house tens of thousands of hogs. Similarly, the dairy industry operates using large factory farms that house thousands of animals in a relatively small land area. While these larger agricultural operations have numerous advantages, attendant with these larger facilities are pollution problems relating to the handling and treatment of manure and wastewater (hereinafter collectively “wastewater”). By way of example, pollution problems associated with liquid animal waste, such as produced by the swine industry, include nitrogen, phosphorus, solids, bacteria and foul odors that result from anaerobic digestion. Environmental concerns more specifically center on odor and ground and surface water quality issue and impacts.

Traditionally, animal wastes and wastewater are collected and stored in waste treatment lagoons or waste storage ponds where they undergo minimal treatment. Most agricultural facilities use microbial digestion for treatment of animal wastes and wastewater. The two primary reasons for using microbial digestion are simplicity and cost. Wastewater is simply discharged from the animal storage facility into an open lagoon or plurality of lagoons (ponds used to store and treat thousands to millions of gallons of animal waste) where the waste undergoes natural microbial digestion. After retention in the lagoon system, wastewater is usually land applied via spray irrigation. However, over forty (40) noxious gases may be emitted from lagoons at hog and/or dairy farms including ammonia, methane and hydrogen sulfide.

Additionally, the time required for digestion of the organic wastes is relatively long, from weeks to months. Some current regulations require a minimum residence of 180 days for animal waste facilities using anaerobic lagoons for digestion. Neighbors find odors emanating from lagoons, confinement houses, and fields onto which wastes are sprayed to be a nuisance. In fact, as a result of odor problems associated with anaerobic lagoons, certain states have legally mandated buffer zones or designated land areas between lagoon sites and populated areas.

The lagoons may be aerobic, anaerobic, or a combination. Anaerobic lagoons, or those requiring the exclusion of oxygen, are good at breaking down solids. However, they are also septic, and emit a very strong odor. Aerobic lagoons, or those requiring oxygen, if operating properly, do a more complete job than anaerobic lagoons of breaking down solids and keep them in suspension longer so there is less residue in the lagoon; harsh odors are also reduced drastically. Further, aerobic digestion is typically quicker than anaerobic.

Most dairy lagoons are not designed to be either anaerobic nor aerobic, they are mainly a storage unit, the top 3 to 6 feet being naturally aerobic because of wind and exposed surface area, while 6 feet and below is more anaerobic due to lack of oxygen. Where both the aerobic and anaerobic populations are healthy, strong, and comprising microorganisms able to process animal waste, the animal waste may be processed very quickly as the aerobic and anaerobic populations may interact in ways which enhance the effectiveness and/or efficiency of the lagoon.

Dairymen spend considerable funds each year putting in aerators and circulators to get air into the lagoons, as well as drudging out the solids that accumulate on the bottom of the lagoon. Some dairies, due to lack of space, have smaller lagoons. In such cases maintenance costs increase significantly. The dairy may end up drudging every three to four years at thousands of dollars every time. Further, because of chemicals used on dairies in animal production, for example chemicals used in foot baths, often this residue is too toxic to be applied to fields without further processing.

Additionally, production areas such as dairy parlors accumulate animal waste during use. While the waste may be moved out of the production area, for example wherein the waste is sprayed with water and allowed to flow out of the production area and into a lagoon, it is typical for a slime to accumulate in the production areas. The slime increases the difficulty of maintaining a clean environment inside the production area, thereby increasing the risk of infection for animals. Further, where the slime accumulates on a floor, the floor may become dangerously slippery. Still further, the slime may generate offensive odors.

While microorganisms may be introduced into production areas, lagoons, farms, and other systems in various ways, it is expensive to purchase, transport and apply such bacteria, in particular where there is a need for a regular supply of the microorganisms to be used in an ongoing operation. While there are methods and products for applying microorganisms; the methods and products are typically inefficient, expensive, complicated, and difficult to maintain. They may include many moving parts, multiple systems, paths, controls, pumps, etc. They may be designed for large facilities and may be very difficult and/or expensive to produce, operate, maintain, and/or change. Further, they may disperse the microorganisms in an inefficient fashion, wasting a significant portion of eh microorganisms. Additionally, while the natural micro-flora found in and around ecosystems is capable of processing agricultural waste and/or enhancing vegetation growth, such micro-flora typically works very slowly, in insufficient amounts and with undesired by-products.

What is needed is a biological reproduction device and/or system that solves at least one of the problems heretofore discussed.

### SUMMARY OF THE INVENTION

The present invention has been developed in response to the present state of the art, and in particular, in response to the problems and needs in the art that have not yet been fully solved by currently available biological reproduction devices and systems. Accordingly, the present invention has been developed to provide a regeneration tank.

In one embodiment, there may be a microorganism regeneration system for providing a regenerating supply of microorganisms for use external to the system. The embodiment may include a hollow vessel, a circulation system, an inlet system, an outlet system, a microorganism quantity, a nutrient quantity, and/or a water quantity. The embodiment may be simply designed with a minimum of parts, especially moving parts. The embodiment may include portions that may encompass one of many of the aforementioned possibly included parts and/or systems.

The hollow vessel may be configured to contain liquids; the circulation system may be disposed within the hollow vessel and configured to circulate and aerate liquid; the inlet system may be coupled to the hollow vessel and configured to cause entry of an aqueous liquid into the hollow vessel only when a liquid volume is below a determined threshold; the outlet system may be coupled to the hollow vessel and configured to controllably permit discharge of liquid from the hollow vessel; the microorganism quantity may be disposed within the hollow vessel, and may have at least a first generation of microorganisms; the nutrient quantity may be disposed within the hollow vessel, and may be in an amount sufficient to sustain reproduction of the microorganism quantity for a reproduction period; the water quantity may be disposed within the hollow vessel, and may be in an amount sufficient to cause the liquid volume to be at the determined threshold.

The circulation system may include a submersible pump configured to circulate liquid within the hollow vessel, wherein the submersible pump is disposed within the hollow vessel and within the liquid volume. The hollow vessel may have an internal volume from about 5 gallons to about 2100 gallons. The liquid volume may have a maximum depth of 72 inches. The circulation system may include a venturi aerator configured to inject air containing oxygen into liquid contained within the circulation system. The hollow vessel may be proximate a plant irrigation system and the outlet system is selectably hydrodynamically coupled to the irrigation system. The circulation system may provide pressure for discharge of liquid through the outlet system when the outlet system permits discharge of liquid from the hollow vessel.

In another embodiment there may be a microorganism regeneration apparatus for regeneration of a supply of microorganisms for use external to the tank. The microorganism regeneration apparatus may include a hollow vessel, a circulation system, an inlet system, and an outlet system. The hollow vessel may be configured to contain liquids; the circulation system may be disposed within the hollow vessel and configured to circulate and aerate liquid; the inlet system may be coupled to the hollow vessel and configured to cause entry of an aqueous liquid into the hollow vessel only when a liquid volume is below a determined threshold; the outlet system may be coupled to the hollow vessel and configured to controllably permit discharge of liquid from the hollow vessel.

In still another embodiment, there may be a method of making and applying microorganisms. The method may include: providing a container proximate a locus of application; including within the container: a first generation of microorganisms; a nutrient quantity; and a water quantity; circulating contents of the container; aerating the contents of the container; extracting a generally known amount of the contents; applying an extracted amount to a locus of application; and/or providing sufficient additional water, nutrients, circulation, and aeration to allow reproduction of organisms sufficient to substantially compensate for an amount previously extracted.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### BRIEF DESCRIPTION OF THE DRAWINGS

In order for the advantages of the invention to be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 illustrates a perspective side view of a regeneration tank and system according to one embodiment of the invention;

FIG. 2 illustrates a cross-sectional side view of a regeneration tank and system according to one embodiment of the invention;

FIG. 3 illustrates a perspective view of an aerator of a regeneration tank and system according to one embodiment of the invention;

FIG. 4 illustrates a cross-sectional side view of a regeneration tank and system according to one embodiment of the invention;

FIGS. 5A and 5B illustrate a system configured to introduce microorganisms into a plant ecosystem according to one embodiment of the invention;

FIG. 6 illustrates a system configured to introduce microorganisms into a plant ecosystem according to one embodiment of the invention;

FIG. 7 illustrates a side view of a dairy cow in a stall in a dairy milking parlor according to one embodiment of the invention;

FIG. 8 illustrates a planar top view of a system configured to introduce microorganisms into a dairy milking parlor and accompanying lagoon according to one embodiment of the invention; and

FIG. 9 illustrates a side cross-sectional view of a dairy milking parlor and accompanying lagoon according to one embodiment of the invention.

### DETAILED DESCRIPTION OF THE INVENTION

For the purposes of promoting an understanding of the principles of the invention, reference will now be made to the exemplary embodiments illustrated in the drawings, and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

Reference throughout this specification to “one embodiment,” “an embodiment,” or similar language means that a particular feature, structure, or characteristic described in connection with the embodiment is included in at least one embodiment of the present invention. Thus, appearances of the phrases “one embodiment,” “an embodiment,” and similar language throughout this specification may, but do not necessarily, all refer to the same embodiment, different embodiments, or component parts of the same or different illustrated invention. Additionally, reference to the wording “an embodiment,” or the like, for two or more features, elements, etc. does not mean that the features are related, dissimilar, the same, etc. The use of the term “an embodiment,” or similar wording, is merely a convenient phrase to indicate optional features, which may or may not be part of the invention as claimed.

Each statement of an embodiment is to be considered independent of any other statement of an embodiment despite any use of similar or identical language characterizing each embodiment. Therefore, where one embodiment is identified as “another embodiment,” the identified embodiment is independent of any other embodiments characterized by the language “another embodiment.” The independent embodiments are considered to be able to be combined in whole or in part one with another as the claims and/or art may direct, either directly or indirectly, implicitly or explicitly.

Finally, the fact that the wording “an embodiment,” or the like, does not appear at the beginning of every sentence in the specification, such as is the practice of some practitioners, is merely a convenience for the reader's clarity. However, it is the intention of this application to incorporate be reference the phrasing “an embodiment,” and the like, at the beginning of every sentence herein where logically possible and appropriate.

Within this specification, including the claims, the phrase “first generation of microorganisms,” and the like, is defined to include the entire set of microorganisms initially present in the biological composition when the biological composition is first put to use as described in the specification, including those that may be reproductive descendants of others present within the biological composition at the same time.

Within this specification, including the claims, the phrase “subsequent generation of microorganisms,” and the like, is defined to include a generation of microorganisms which are reproductive descendants, whether immediate or not, of the first generation of microorganisms.

Within this specification, including the claims, the phrase “continuing generation of microorganisms,” and the like, is defined to include a generation of microorganisms which are reproductive descendants, whether immediate or not, of the subsequent generation of microorganisms.

Within this specification, including the claims, the phrase “microorganism quantity” is defined to include the microorganisms of the biological composition, including all reproductive descendants. Further it includes any and all other material which may be present with the microorganisms as they are mixed together with other materials to form the biological composition. These materials may include inert carrier materials as well as any other materials.

Within this specification, including the claims, the term “vegetation,” and the like, is defined to include all plants and other flora. Within this specification, including the claims, the term “animal waste,” and the like, is defined to include all waste associated with animal production.

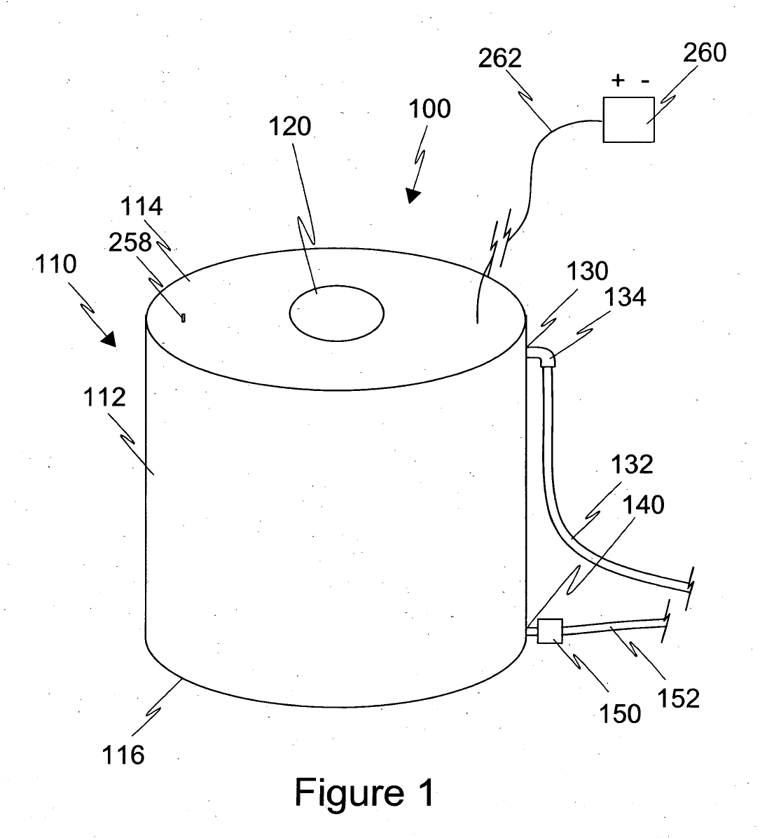

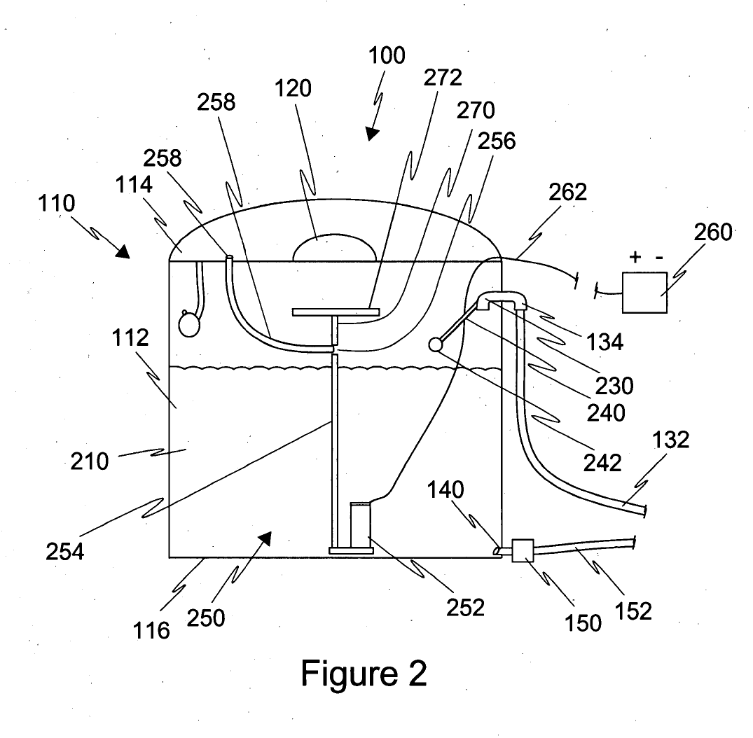

Looking to FIGS. 1-3, there may be a regeneration tank 100 according to one embodiment of the invention. The regeneration tank 100 may include an inlet system, an outlet system, and/or a circulation system. Portions of the systems are described further herein. Such a regeneration tank 100 for producing microorganisms may include a hollow vessel 110 that can be defined by an enclosing wall 112, a top 114, and a base 116. Additionally, the vessel can be configured to hold a liquid 210 that may be a liquid mixture of water, microorganisms, and nutrients. The hollow vessel 110 may have a predetermined liquid level that may be a maximum liquid level. The liquid level may vary in accordance with different aspects of the invention. In an additional aspect, a volume of the hollow vessel 110 may vary. In one aspect, the volume may be less than about 2100 gallons. In another aspect, the volume may be greater than about 5 gallons. In still another aspect, the hollow vessel 110 may include a lid opening 120 that may be configured to receive a lid. It may be that the lid may be removable and/or that the lid may be adapted to seal the lid opening 120. There may be a vent (not shown) configured to simultaneously permit air exchange and restrict entry of solid and/or liquid materials into the hollow vessel 110.

The hollow vessel 110 may include an inlet opening 130 that may be hydrodynamically coupled to an intake valve 230 configured to regulate a flow of water, preferably fresh water, or other liquid, into the hollow vessel 110. The intake valve 230 may be of any type, including but not limited to solenoid, ball, and/or float valves. The intake valve 230 may selectably-hydrodynamically couple the hollow vessel 110 to an inlet hose 132. There may be an inlet hose coupling 134 that may hydrodynamically couple the inlet hose 132 to the hollow vessel 110, inlet opening 130, and/or intake valve 230. The inlet hose 132 may be hydrodynamically coupled to a source of liquid, such as a supply of irrigation water.

There may be a liquid level regulator 240 that may be mechanically coupled to the hollow vessel 110 and may be configured to control the liquid level within the hollow vessel 110. The liquid level regulator 240 may be functionally coupled to the intake valve 230. The liquid level regulator may include a float 242 for detecting and controlling liquid level. Accordingly, the liquid level regulator 240 may be configured to stop a flow of liquid from entering the hollow vessel 110 through the inlet opening 130. The liquid level regulator 240 may be configured to control the intake valve 230 when the liquid level reaches a predetermined level. Such a liquid level regulator 240 may include a float shutoff valve.

There may be an outlet opening 140 that may be proximate to the base 116. The outlet opening 140 may be hydrodynamically coupled to an outlet valve 150 configured to regulate a flow of liquid out of and from the hollow vessel 110. The outlet valve 150 may selectably-hydrodynamically couple the hollow vessel 110 to an outlet hose 152. There may be an outlet hose coupling configured to couple the outlet hose 152 to the hollow vessel 110, outlet opening 140, and/or outlet valve 150. There may be a storage tank (not shown) coupled to the hollow vessel 110 by the outlet hose 152. The storage tank may be configured to mix and/or store liquid for later use.

There may be included within the hollow vessel 110 a circulation system 250 configured to circulate liquid within the hollow vessel 110. The circulation system 250 may be configured to transport liquid from proximate to the base 116 of the hollow vessel 110 to a predetermined height that may be above the surface of the predetermined liquid level. There may be a pump 252 configured to pump liquid within the hollow vessel 110. The pump 252 may be configured to be submersible and may be disposed within the hollow vessel 110, preferably below the liquid level. The pump 252 may be electrically coupled to a power supply 260, preferably by an appropriately insulated power cord 262.

Within the circulation system 250, there may be a transport tube 254 configured to facilitate transportation of liquid within the hollow vessel 110. The transport tube 254 may be configured to dispose liquid within the hollow vessel 110 but above the liquid level, thereby exposing the disposed liquid to air. The transport tube 254 may further include an air inlet 256 disposed on the transport tube and configured to permit injection of air into the transport tube. There may be an air intake tube 258 coupled to the air inlet 256 and configured to provide air, preferably containing oxygen (such as normal air does), to the air inlet 256. The air inlet 256 may be a venturi, thereby using pressure generated by the pump 252 to inject air into the circulation system 250. Thereby there may be no need for a separate air pump.

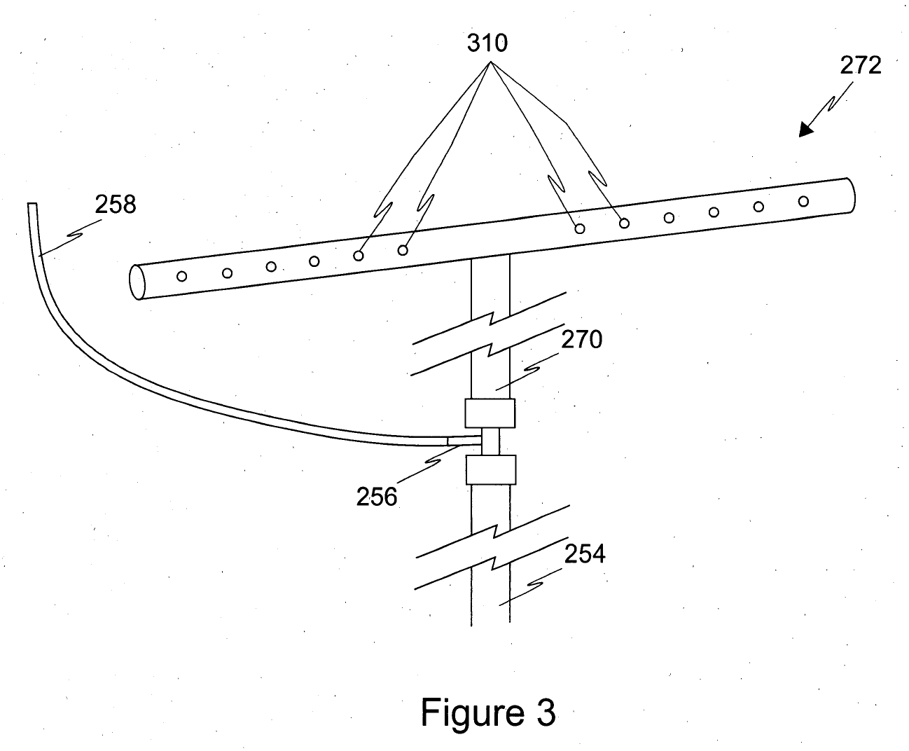

Looking in particular to FIG. 3, there may be a second end 270 of the transport tube 254 that may include a spout assembly 272 configured to spray liquid over the surface of the liquid within the hollow vessel 110. There may be a spout having at least one hole or spout outlet 310 configured to permit release of liquid from the transport tube 254.

In operation, the regeneration tank 100 holds water within the hollow vessel 110. There is disposed therein a quantity of microorganisms and an associated quantity of nutrients. The circulation system 250 causes circulation and aeration beneficial to the reproduction of the microorganisms as the microorganisms consume the nutrients. As quantities of microorganisms are generated, a user may discharge portions of the mixture for use. For example, a farmer may discharge liquid from the regeneration tank into an irrigation system at a constant rate. As another example, a dairy farmer may discharge a set quantity of liquid over a production area in a short period of time each day.

As portions of the mixture are discharged, the quantities of microorganisms and water are reduced. Water may be introduced through the inlet 130 to replace water volume discharged. As the microorganisms are circulated through the liquid in the hollow vessel and are subjected to an environment facilitative of reproduction, the amounts of microorganisms regenerate, replacing quantities discharged. Thereby the regeneration tank regenerates quantities of microorganisms in liquid.

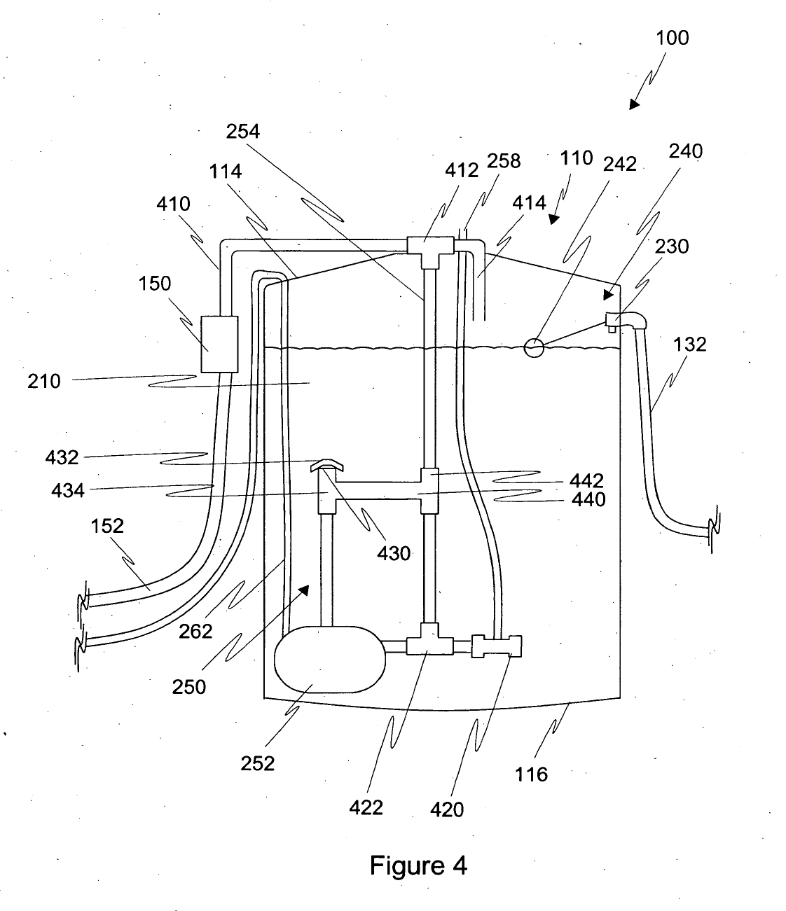

FIG. 4 illustrates a cross-sectional side view of a regeneration tank 100 and system according to one embodiment of the invention. There may be a regeneration tank 100 according to one embodiment of the invention. The regeneration tank 100 may include an inlet system, an outlet system, and/or a circulation system. Portions of the systems are described further herein.

Such a regeneration tank 100 for producing microorganisms may include a hollow vessel 110 that can be defined by an enclosing wall 112, a top 114, and a base 116. Preferably the hollow vessel 110 is constructed of plastic and/or stainless steel, as many microorganisms are capable of destroying many other materials. Also, it is preferred that the hollow vessel 110 be opaque to reduce algae growth within the tank. Further, it is preferred that the hollow vessel be a light color, such as white, blue or yellow, as darker tanks, such as black tanks, absorb a lot of heat, thereby often reducing efficiency and/or regeneration rate by increasing the temperature of a liquid 210.

The hollow vessel 110 can be configured to hold a liquid 210 that may be a liquid mixture of water, microorganisms, and nutrients. The hollow vessel 110 may have a predetermined liquid level that may be a maximum liquid level. The liquid level may vary in accordance with different aspects of the invention. In an additional aspect, a volume of the hollow vessel 110 may vary. In one aspect, the volume may be less than about 2100 gallons. In another aspect, the volume may be greater than about 5 gallons. Preferably, the volume will be less than about 1550 gallons as larger volumes are more difficult to properly oxygenate, especially in lower sections of tanks. Preferably the depth of liquid within the hollow vessel 110 does not exceed 72 inches else the water pressure causes difficulties in having proper amounts of dissolved oxygen. More preferred, the depth of liquid within the hollow vessel 110 does not exceed 36 inches.

The hollow vessel may include an inlet system. The inlet system may include one or more of the following characteristics and/or parts. Further, it is understood that the inlet system may include other characteristics and/or parts and that a characteristic and/or part of an inlet system may simultaneously be a part and/or characteristic of another system. The hollow vessel 110 may include an intake valve 230 configured to regulate a flow of water, preferably fresh water, or other liquid, into the hollow vessel 110. The intake valve 230 may be of any type, including but not limited to solenoid, ball, and/or float valves. The intake valve 230 may selectably-hydrodynamically couple the hollow vessel 110 to an inlet hose 132. The inlet hose 132 may be hydrodynamically coupled to a source of liquid, such as a supply of irrigation water.

There may be a liquid level regulator 240 that may be mechanically coupled to the hollow vessel 110 and may be configured to control the liquid level within the hollow vessel 110. The liquid level regulator 240 may be functionally coupled to the intake valve 230. The liquid level regulator 240 may include a float 242 for detecting and controlling liquid level. Accordingly, the liquid level regulator 240 may be configured to stop a flow of liquid from entering the hollow vessel 110 through the intake valve 230. The liquid level regulator 240 may be configured to control the intake valve 230 when the liquid level reaches a predetermined level. Such a liquid level regulator 240 may include a float shutoff valve.

There may be included within the hollow vessel 110 a circulation system 250 configured to circulate liquid within the hollow vessel 110. The circulation system 250 may include one or more of the following characteristics and/or parts. Further, it is understood that the circulation system 250 may include other characteristics and/or parts and that a characteristic and/or part of a circulation system 250 may simultaneously be a part and/or characteristic of another system. The circulation system 250 may be configured to transport liquid from proximate to the base 116 of the hollow vessel 110 to a predetermined height that may be above the surface of the predetermined liquid level. There may be a pump 252 configured to pump liquid within the hollow vessel 110. The pump 252 may be configured to be submersible and may be disposed within the hollow vessel 110, preferably below the liquid level. Submersion of the pump 252 within the hollow vessel 110 advantageously provides a heat sink for the pump 252 and protects the pump from outside elements and damage. The pump 252 may be electrically coupled to a power supply 260, preferably by an appropriately insulated power cord 262.

Within the circulation system 250, there may be a transport tube 254 configured to transport liquid substantially vertically within the hollow vessel 110. The transport tube 254 may be configured to dispose liquid within the hollow vessel 110 preferably above the liquid level, thereby exposing the disposed liquid to air.

There may be an air tube 258 for permitting air including oxygen (such as that included in normal air) to be accessed by the liquid 210. Preferably, the air tube 258 is coupled to an aerator 420 configured to expose the liquid 210 to air from the air tube 258. The aerator 420 may be coupled to the circulation system 250, preferably at an aerator junction 422. Preferably air may be injected into the circulation system 250 at a ratio of about 1 gallon of air per about 36 gallons of water being circulated. The aerator 420 may be a venturi aerator operated by the pump 252. In one aspect the aerator 420 may inject air into the liquid 210 at a rate of about 1:16 parts air to parts water. In another aspect the aerator 420 may inject air into the liquid 210 at a rate of about 2:16 parts air to parts water.

The circulation system 250 may also include a circulation intake 430 for permitting liquid 210 to enter the circulation system 250. The circulation preferably includes an intake screen 432 for preventing solids from entering the circulation system 250. The circulation intake 430 may be coupled to the circulation system 250 by an intake junction 434. Preferably the circulation intake 430 is near the base 116 of the hollow vessel 110, thereby enhancing circulation of the liquid 210. Preferably the circulation intake 430 is within 18 inches of the base 116 of the hollow vessel 110. The circulation intake 430 may be hydrodynamically coupled to the pump 252 and may be disposed adjacent to the pump 252, thereby enhancing liquid flow around the pump 252 and aiding in cooling the pump 252. For example, the circulation intake 430 may be disposed vertically above the pump 252 and within about 1 inch to about 18 inches away from the pump 252.

There may be a pipe brace 440 and 442 configured to mechanically couple the transport tube 254 to the circulation intake 430, thereby providing enhanced stability for both. There may be additional braces, straps, and/or other stability enhancing devices configured to provide-stability to the transport tube 254 and/or the circulation intake 430.

The hollow vessel may include an outlet system. The outlet system may include one or more of the following characteristics and/or parts. Further, it is understood that the outlet system may include other characteristics and/or parts and that a characteristic and/or part of an outlet system may simultaneously be a part and/or characteristic of another system. There may be an outlet tube 410 for discharging liquid from the regeneration tank 100. The outlet tube 410 may be hydrodynamically coupled to the circulation system 250 by an outlet junction 412, thereby permitting water pressure generated by the circulation system 250 to facilitate discharge of fluids from the regeneration tank 100. The outlet junction 412 may hydrodynamically couple to the circulation system 250 at the transport tube 254. The outlet tube 410 may be hydrodynamically coupled to an outlet valve 150 for controlling discharge of fluids from the regeneration tank 100. The outlet valve 150 may be hydrodynamically coupled to an outlet hose 152 for directing discharged fluids. The outlet valve 150 may permit selectable hydrodynamic coupling between the outlet tube 410 and the outlet hose 152. The pump 252 may be hydrodynamically coupled to the aerator 420 and/or the outlet tube 410, thereby permitting a single pump 252 to perform three duties. Advantageously, the configuration of the regeneration system may be simplified, making the system more efficient, less expensive, less difficult to produce, less difficult to setup, less expensive to maintain, and/or less likely to malfunction.

There may also be a circulation outlet 414 for releasing fluid from the circulation system 250 back into the liquid 210. The circulation outlet 414 may hydrodynamically couple to the transport tube 254. For example, the circulation outlet 414 may be coupled to the transport tube 254 by the outlet junction 412. Therefore, water pressure from the circulation system 250 may cause fluid to exit the circulation system 250 through the circulation outlet 414 and back into the liquid 210, thereby circulating the liquid 210, thereby providing enhanced reproductive conditions for microorganisms therein. The circulation outlet 414 may release fluid from the circulation system 250 back into the liquid 210 by releasing fluid into air above the liquid 210, thereby enhancing aeration of the liquid 210 by permitting additional air contact with the released fluid, disrupting formed membranes on the liquid 210 surface, and causing turbulent flow into the liquid 210.

In operation, the hollow vessel 110 may have a liquid 210 disposed therein including microorganisms and nutrients. The circulation system 250 may circulate and aerate the liquid contents of the hollow vessel, thereby enhancing the reproductive capacity of the microorganisms and reducing a possibility that harmful/unwanted odors may be produced. Preferably, wherein there may be a daily discharge of from about 75% to about 90% tank volume of liquid 210 there may be circulation of the tank about once each hour. As the microorganisms reproduce t6 a desired amount or concentration, the outlet valve 150 may selectably permit discharge of liquid 210 from the hollow vessel 110, thereby permitting use of the concentrated microorganisms.

Discharge of liquid 210 from the hollow vessel 110 may be compensated for by the intake valve 230 permitting additional liquid, such as water, to be disposed inside the hollow vessel 110. Additional water may reduce the concentration of microorganisms, which then may reproduce to a desired concentration. Thereby the regeneration tank 100 may regenerate liquid concentrations of microorganisms.

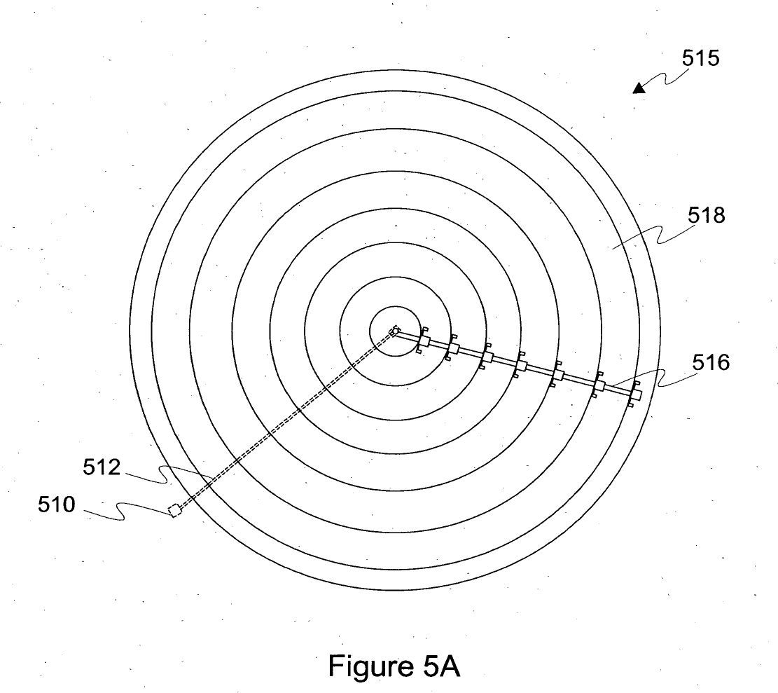

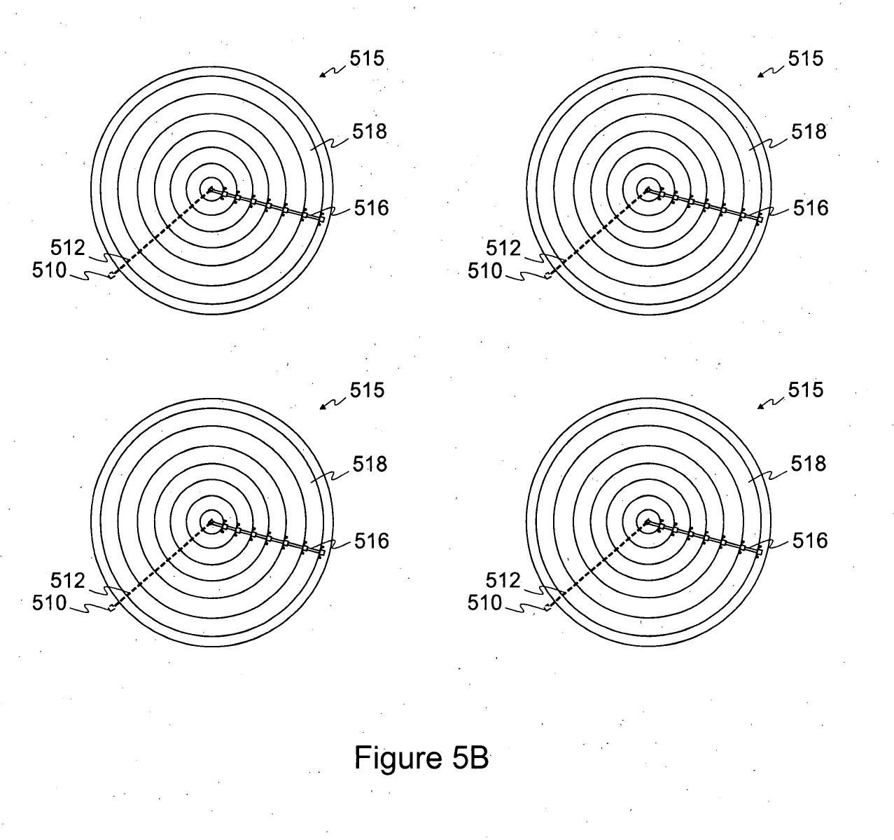

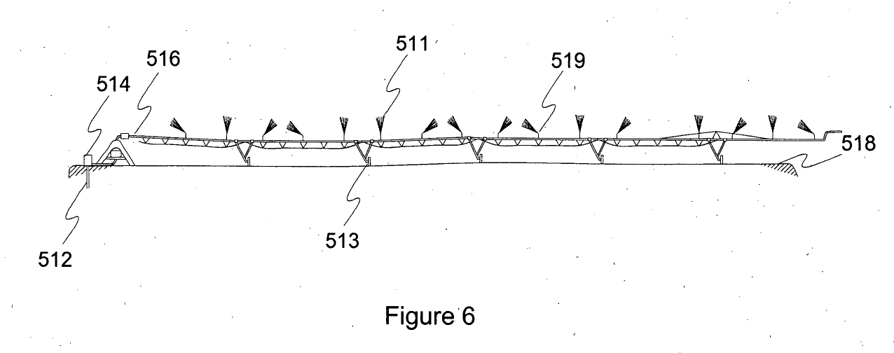

FIGS. 5A, 5B, and 6 illustrate a system configured to introduce microorganisms into a plant ecosystem according to one embodiment of the invention. There is a tank, or container 510, connected to a feed pipe 512, containing irrigation water and extending underground, of an irrigation system 515 configured to irrigate a plant ecosystem 518. While the container 510 is shown to be a significant distance from the center of the irrigation system 515, it is understood that the container 510 may be adjacent to the center of an irrigation system 515 and connected by a much shorter appropriate feed pipe 512. Also, a single container 510 may serve multiple irrigation systems 515. The irrigation system 515 further includes a junction 514 connecting the feed pipe 512 to the irrigation pipe 516. The irrigation pipe 516 includes sprinklers 519 configured to distribute fluid over the plant ecosystem 518. Further, there are wheels 513 configured to permit displacement of the irrigation pipe 516 about the plant ecosystem 518.

In operation, microorganisms are generated, or regenerated, as products and further generations of a biological composition, in a liquid mixture in the container 510. The feed pipe 512 may supply water to the container 510 as needed. The liquid mixture is introduced into the feed pipe 512 at a known rate or at known portions per elapsed time. Preferably, the rate at which the liquid mixture is introduced into the feed pipe 512 does not exceed the rate at which the liquid mixture is generated by the container 510. For example, where the container generates, or regenerates, 100 gallons of liquid mixture per day, it is preferable that the rate at which the liquid mixture is introduced, or injected, into the feed pipe does not exceed 100 gallons per day, regardless of whether the rate is constant throughout a twenty-four hour period. The liquid mixture portions mix with the irrigation water as the irrigation water flows through the feed pipe 512, through the junction 514, through the irrigation pipe 516, through the sprinklers 519, which sprinklers distribute the combined fluids 511 over the plant ecosystem 518.

As illustrated in FIG. 5B, there may be multiple irrigation systems 515 each configured to irrigate an area. Each irrigation system 515 may have a tank 510 configured to introduce a liquid mixture of a biological composition into the irrigation system 515 for enhancing vegetation growth.

Microorganism generation, and/or regeneration, consumes nutrients. Further, generations of microorganisms tend to mutate, or change, from strains originally introduced. Therefore, preferably, nutrients and original strains of microorganisms are introduced into the container as needed. For example, biological compositions, preferably in pre-made packets including a nutrient quantity and a quantity of original strain(s), or microorganism quantity, are preferably placed within the container on a regular schedule, such as once a week.

In operation of one embodiment, the biological composition may provide a two stage rich source of food and other nutrients for the quantity of microorganisms. The first stage permits rapid reproduction of the microorganism. This beneficially permits the microorganism to predominate other competing microorganisms and creates a large quantity of microorganisms in a short period of time, which then may be dispersed into a plant ecosystem. The second stage facilitates maintenance of the microorganism population as portions are extracted and dispersed into a plant ecosystem. During both stages, important nutrients are supplied by the biological composition to the reproducing microorganism population.

Additionally, in operation of one embodiment, the two stage rich source of food is configured to last for a reproduction period, with the materials providing the first stage being substantially consumed before the expiration of the reproduction period. For example, the biological composition may be configured to last for a week, with the first stage configured to be substantially consumed within 48 hours. In another example, the biological composition may be configured to last 4 days, with the first stage configured to be substantially consumed within 24 hours.

One skilled in the art would know that by varying the proportions and total amounts of the materials comprising the ingredients of the two stages and the initial microorganism content of the biological composition, one may adjust the biological composition to generally conform the reproduction period to any reasonable desired period of time. Also, it is not necessary that the reproduction period be known to the manufacturer or that it be designated at all, merely that there be a period of time to which the composition relates as described herein.

Also, in operation of an embodiment, oxygen is introduced to a liquid mixture containing microorganisms in a container. This may be accomplished by injection, bubbling, interface exchange, or any other method known in the art for providing access to oxygen within a liquid. The source of oxygen may be normal air.

Still yet, in operation of one embodiment, portions of a liquid mixture containing microorganisms in a container are extracted from the container. This may be accomplished with an outlet, preferably coupled to a lower portion of the container to avoid clogging with any surface skins formed on a top surface of the liquid mixture in the container.

In addition, the nutrient quantity preferably includes a food source easily utilized by the microorganisms, or short-term nutrient, to promote rapid generation of microorganisms, in particular rapid generation of the original strain. Additionally, the nutrient quantity preferably includes a long lasting food source, or long-term nutrient, configured to nourish the microorganisms after the short-term food source depletes. Also, the nutrient quantity preferably includes a quantity of other nutrients, or supplement nutrients. The nutrient quantity may include but is not limited to vitamins, minerals, enzymes, amino acids, protein compositions, starches, fibers, carbohydrates, sugars, growth media, proteins, chelating agents, complexing agents, sequestering agents, and other materials useful in nourishing microorganisms and plants.

In particular, it is preferred that the microorganism quantity includes a microorganism(s) characterized by the ability to enhance vegetation growth, preferably in a plant dominated ecosystem such as a field of human cultivated plants. Examples of beneficial microorganisms include but are not limited to bacteria, yeasts, protozoa, actinomycites, and nematodes. It is preferred that the microorganism quantity includes a microorganism(s) characterized by the ability to produce cellulase enzymes, to convert nutrients to a form usable by plants, to loosen soil, to enhance water retention in soil, to combat parasitic organisms, and/or to otherwise enhance soil vitality, plant vitality, crop production, plant health, and/or crop production efficiency. Additionally, the microorganisms may be aerobic bacteria. The microorganism(s) may include, but is not limited to bacillus subtilis, bacillus lichenformis, bacillus cereus, bacillus megaterium, fluorescent pseudomonas, azobacter, cellulase enzyme producing bacteria, yeasts, sub-cultures thereof, and combinations thereof.

Still further, it is preferred that the microorganism(s) in the microorganism quantity be included in sufficient quantities to predominate other microorganisms which may use the biological composition to reproduce. “Other microorganisms” as used in the previous sentence may include microorganisms present in a container, in a water supply feeding into a container, strains of similar microorganisms which may have mutated from an original strain related to or identical to the microorganisms present in the microorganism quantity.

Still, in particular, it is preferred that the short-term nutrient include ingredient(s) characterized by the ability to provide a quick and ready source of nourishment for the microorganisms of the microorganism quantity. Preferably, this may include but is not limited to hydrolyzed collagen, bone meal, blood meal, carbon skeleton molecules, sugars, carbohydrates, folvic acid, organic acid, soy protein, peptone treated biological matter (such as peptone treated animal carcasses or peptone treated plant matter), other easily consumed materials and combinations thereof. Preferably, the short-term nutrient is present in the biological composition in sufficient amounts to provide for rapid reproduction of the microorganism quantity and its further generations for a reproduction period.

Further, in particular, it is preferred that the long-term nutrient include ingredients(s) characterized by the ability to provide a stable, lasting (as compared to the short-term nutrient and/or the intended period of replacement of the biological composition packets) source of nourishment for the microorganisms of the microorganism quantity. Preferably, this may include but is not limited to wheat starch, soy flour, molasses, processed or raw animal and/or plant matter, other slowly consumed proteins, fibers, starches, fats and carbohydrates and combinations thereof. Preferably, the long-term nutrient is present in the biological composition in sufficient amounts to provide for continued reproduction of the microorganism quantity and its further generations for a reproduction period after the short term nutrient quantity is substantially consumed.

Again, in particular, it is preferred that the supplement nutrient include ingredient(s) characterized by the ability to provide for the variety of nourishment needs of the microorganisms of the microorganism quantity. It is preferred that the supplement nutrient at least provide for at least one the non-energy source needs of the microorganisms of the microorganism quantity. Preferably, the ingredient(s) of the supplement nutrient may include but is not limited to food grade proteins; vitamins; inorganic salts; amino acids; growth media; minerals such as phosphate, potassium, calcium, sulfur, cobalt, copper, iron, magnesium, sodium, manganese, and zinc; humate and/or humic acids; enzymes; chelating, complexing, and/or sequestering agents with or without associated molecules; and combinations thereof. Preferably, the supplement nutrient is present in the biological composition in sufficient amounts to nourish the microorganism quantity and its further generations for a reproduction period.

Preferably, the biological composition is in a dry form wherein the biological composition may be stored for a time with the microorganisms in an inactive state. Preferably, the biological composition is a package. Preferably, the biological composition may be powder, granules or a pressed cake. In addition, the biological composition is preferably configured to aid or induce generation or regeneration of a quantity of microorganisms, preferably in a container or system configured to introduce microorganisms into a plant ecosystem. Also, preferably, the biological composition is configured to introduce or reintroduce an early generation of the microorganisms into the container or system. “Early generation” means that the microorganisms are not substantially mutated from the desired species, strains, and/or characteristics.

### EXAMPLE ONE

There is a dry microorganism amount which includes base soil bacteria, for example, the product known under the brand name Soil Response™. The product known as Soil Response™, is attributed to SafeWaze at 7411 N. Tryon Street in Charlette, N.C. 28213. The product known as Soil Response™ is a mixture of active hydrocarbon oxidizing, natural single-cell organisms, specifically for use on soil including, but is not limited to Pseudomonas Fluorescent, Azotobacter, as well as Cellulase enzymes producing bacteria. The microbes are contained in an inert preparation of a natural absorbent which has no chemical impact.

There is a dry nutrient amount which includes a micro-nutrient supplement, a long-term food source and a short term food source. The micro-nutrient supplement is manufactured by the organization having the trademark SafeWaze™ at 7411 N. Tryon Street in Charlette, N.C. 28213. It includes a blend of food grade proteins, vitamins, inorganic salts, and growth media—intended as a supplementary food supply for microorganisms. The micro-nutrient package may include, but is not limited to minerals and nutrients including phosphate, potassium, calcium, sulfur, cobalt, copper, iron, magnesium, and zinc, as well as proteins. The long-term food source is produced under the label DRI-MOL®, which is a dry molasses product manufactured by the organization having the trademark ADM found in Stanley, Wis. 54768. The ingredients include molasses, wheat Starch, calcium strearate, soy flour and lecithin. The short-term food source is hydrolyzed collagen, or HC, of type GCP-1000 which is manufactured by the organization known as Nitta Gelatin NA, INC. at 201 W. Passaic St. in Rochelle Park, N.J. 07662.

The composition amounts are detailed below, with a period of usage of seven days and a fresh clean out at the beginning of each season.

Biological Package Composition and Application Usage:

| | | Applied into Container on |
|---|---|---|
| | Density | Days 1, 7, 14, 21 |
| Soil Response TM | 6.25 ounces/cup | 0.625 cups |
| Micro-Nutrient | 6 ounces/cup | 0.125 cups |
| Supplement | | |
| DRI-MOL ® | 5.5 ounces/cup | 2.5 cups |
| Hydrolyzed Collagen HC | 3.25 ounces/cup | 0.75 cups |

### EXAMPLE TWO

| | Approximate Percent by Volume |
|---|---|
| Dry Bacteria Culture | 5% |
| Yeast | 1% |
| Soybean Protein | 18.75% |
| Nutrient Mineral Mix | 18.75% |
| Humate | 18.75% |
| Granulated Sugar | 18.75% |
| Flour | 18.75% |

The nutrient mineral mix included crude protein, crude fat, salt, calcium, chlorine, magnesium, phosphorous, potassium, sodium, sulfur, cobalt, copper, iodine, iron, manganese, and zinc.

### EXAMPLE THREE

Examples of materials which are includable in embodiments include a variety of different biological and food mixes including soy protein use for animal feed supplements, mineral mixes used in animal nutrition, protein packages used in body building and dietary supplement, flours, sugar, raw molasses, yeast, various enzymes used in waste treatment, laundry soaps and the product sold under the mark Oxy Clean. Also included are various combinations of products including the product sold under the marks ViBasic, Xcite and ViPlex from the organization under the mark Vitech Industries. Further examples include the product under the mark Esp333, both liquid and dry, as well as a protein feed supplement from the organization under the mark Bio-Kinetics, soil stimulant from the organization known as Fertile Earth, along with various combinations of fertilizers.

### EXAMPLE FOUR

Using flood irrigation, a farmer raised 25% more alfalfa hay than his prior average using 6.5 gallons of a product of a biological composition as claimed dripped into the first irrigation, 15 gallons in his second irrigation, and 30 gallons in third. On two fields, the treatment so improved water retention that only two irrigations were required to achieve the same yield increase.

### EXAMPLE FIVE

Using pivot irrigation, a farmer farms 80 pivots. Most of the pivots are 125-acre pivots, with a few going up to 160 acres. One pivot is 540 acres. Due to the size of the 540 acre pivot and the time it take for one rotation of the 540 acre pivot the farmer was having problems with it drying out. This affected yields and hay quality. The farmer started applying a product of a biological composition dispersed as claimed through the pivot at a rate of 400 gallons per day. By the time of the first cutting, the hay stand was thicker and the soil was moist. Throughout the course of the year, the fields continued to see soil, yield and quality improvements.

### EXAMPLE SIX

Using pivot irrigation, a farmer had a pivot on a hillside where the high side of the field would not retain water and the alfalfa growth was always shorter. The farmer had tried other products on the field with little results. Use of the products of the biological composition commenced during the heat of the summer when water problems are at a peak. After just a few weeks of using on the products of the biological composition, at the rate of 120 gallons per day, growth on the high side of the field was as good as the rest of the field and yielding the same.

It is understood that the above-described preferred embodiments are only illustrative of the application of the principles of the present invention. The present invention may be embodied in other specific forms without departing from its spirit or essential characteristics. The described embodiment is to be considered in all respects only as illustrative and not restrictive. The scope of the invention is, therefore, indicated by the appended claim rather than by the foregoing description. All changes which come within the meaning and range of equivalency of the claims are to be embraced within their scope.

It is also understood that the words “irrigate” and “irrigation” are not limited to one or several means to provide water, such as flood irrigation or pivot irrigation. Unless otherwise indicated, the terms “irrigate” and “irrigation” and similar terms are intended to indicate any means or act of providing with water or other similar fluid.

Also, although the figures illustrate a particular irrigation system and growth container, it is understood that not all details of the configuration and/or conjunction are shown. Also, it is envisioned that the invention may be adapted to couple with any system for dispersing water into a plant ecosystem, or that the invention may be an integral part of such system.

Additionally, although the figure and examples illustrate specific compositions, it is understood that the scope of the invention is to be determined by the claims and not by the specific examples.

It is also envisioned that one embodiment may include one material as a short-term nutrient while another may include that same material as a long-term nutrient, depending on the characteristics of the microorganisms in the microorganism quantity and/or the intended period of packet replacement.

One skilled in the art would know that although the invention is sometimes expressed as a mixture of quantities, it is not necessary that the quantities be distinct. For example, the same ingredient forming the short-term nutrient quantity may also supply at least some of the supplement nutrient quantity, as may be the case where a short-term nutrient quantity includes hydrolyzed collagen.

It is expected that there could be numerous variations of the design and/or configuration of embodiments of this invention. An example is that the biological composition may include additional materials beyond those disclosed. These materials may be carrier materials, inert materials, granulation materials, caking materials, or may or may not serve another function such as a beneficial function related to the packaging, shipping, storing, manufacturing, usage, and/or compliance-with authority needs.

Also, it is envisioned that the embodiments of the invention may be constructed of a variety of materials. It is envisioned that the embodiments of the invention may include materials, not herein disclosed but which may be known in the art, having properties substantially similar to those which have been disclosed.

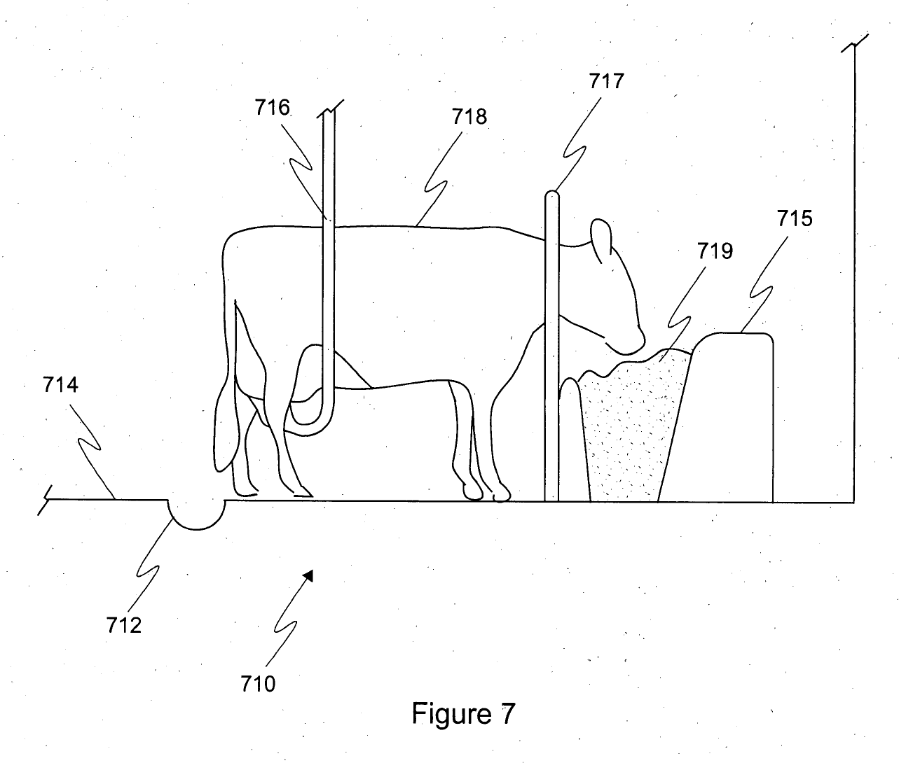

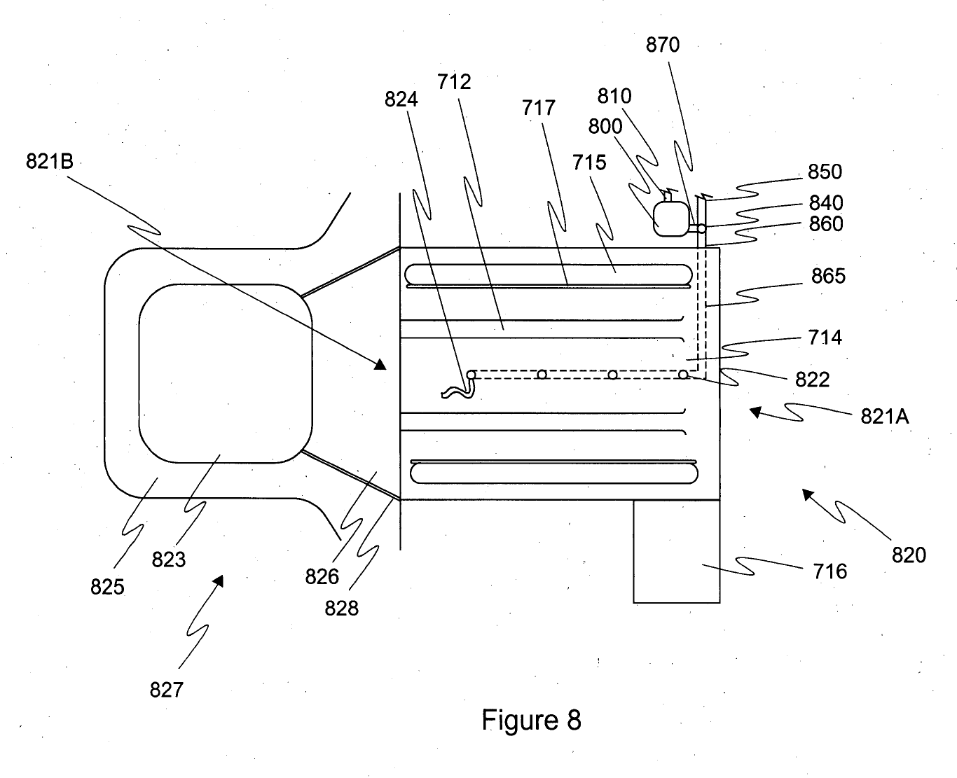

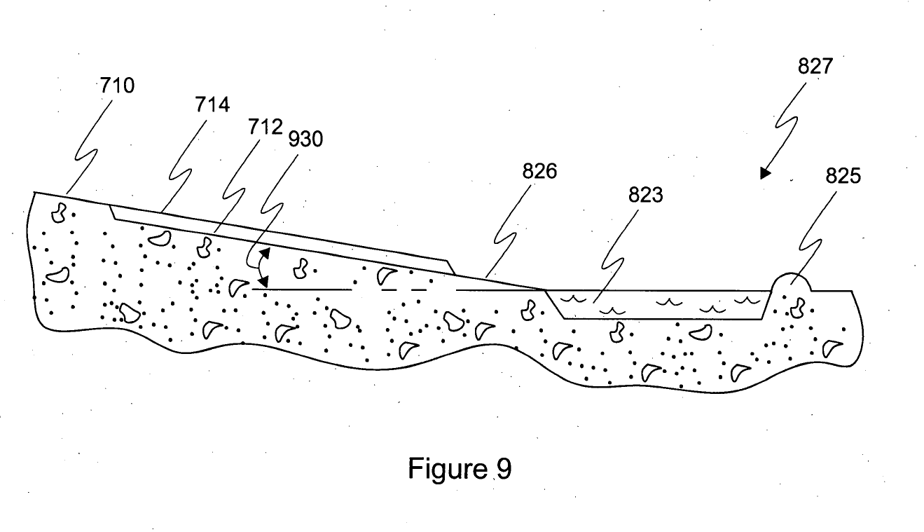

FIGS. 7-9 illustrate a system configured to introduce microorganisms into a dairy milking parlor and accompanying lagoon according to one embodiment of the invention. FIG. 7 illustrates a side view of a dairy cow in a stall in a dairy milking parlor according to one embodiment of the invention. FIG. 8 illustrates a planar top view of a system configured to introduce microorganisms into a dairy milking parlor and accompanying lagoon according to one embodiment of the invention. FIG. 9 illustrates a side cross-sectional view of a dairy milking parlor and accompanying lagoon according to one embodiment of the invention.

There is illustrated an animal production facility that may be a dairy milking parlor, feed parlor, or parlor 820 having an animal entrance/exit 821A wherein animals 718, such as dairy cows 718, may enter or leave the parlor and a waste exit 821B configured to permit the exit of fluid or fluid-like waste material from the parlor 820. For convenience, both feed and milking elements are included in the drawings although typically milking and feeding functions are performed within separate facilities. A parlor floor 710 may be configured to be generally sloping downward towards the waste exit 821B (as generally shown in FIG. 9) to create a natural tendency for fluid waste to travel out of the parlor 820 by way of the waste exit 821B.

Preferably, the dairy milking parlor 820 includes milking apparatus 716 used in extracting milk from animals 718. Also, preferably the parlor 820 includes stalls configured to organize production of the animals 718. In particular, there may be feed troughs 715 configured to be accessed by the animals 718 in each stall through rails 717, wherein the feed troughs 715 may contain animal food 719. Further, the milking apparatus 716 may be configured to extract milk from the animals 718 while the animals 718 are organized in the stalls.

Preferably, there are waste flush alleys 712, or flush alleys, positioned to catch animal waste from each of the stalls as the animals 718 are being processed by the milking apparatus 716. The waste flush alleys 712 may be sloped similarly to the parlor floor 710 and configured to contain fluid waste and direct the fluid waste out of the parlor 820 by way of the waste exit 821B. The flush alleys 712 may run the entire length of the parlor 820 and may be as narrow or wide as is appropriate and as shallow or deep as appropriate. Further, there may be a retaining wall (not shown) defining boundaries of the flush alley 712. Also, there may be grooves (not shown) running the length of the flush alley 712.

Further, there is preferably a parlor cleaning water system configured to provide water for spraying surfaces in the parlor 820, especially the parlor floor 710. There may be hose attachment spigots 822 included in the parlor cleaning water system configured to provide sources of water for a hose 824, which may then be used to distribute water for cleaning the parlor 820. The source of water may be fresh water, for example, for use in cleaning a milking parlor, or may be water from a second lagoon in a multi-stage lagoon system, for example, for use in a feed parlor. Preferably, there is also a microorganism-generation tank 700 in regulatable fluid communication with the parlor cleaning water system, preferably by means of a feed pipe 870. The microorganism-generation tank 700 may be outside the facility or may be enclosed within the facility for protection from the elements. Preferably, there is a lagoon system, or lagoon 827 in general proximity to the parlor 820. Further, it is preferred that the lagoon system 827 be in communication with the parlor 820 such that fluid exiting the parlor may be directed to the lagoon 827. The lagoon 827 provides a reservoir for containing fluid waste during the time required for waste processing. The lagoon system 827 may comprise multiple lagoons 827. For example, there may be a first lagoon configured to break down solid waste and a second lagoon configured to further process the waste and act as a storage lagoon.

Typically the lagoon 827 is usually a 2 lagoon system. The first lagoon processes the waste and the second is for storage and finishing. Most recycled flush water comes from the second lagoon. After being washed down the flush alley 712, wastewater usually goes through a separator that removes most large solids and the removed solids are taken for compost, this removes anywhere from 50% to 80% of the solids. However, some dairies use only one lagoon and others may have three or more lagoons. Lagoon sizes may typically vary between 3 to 15 feet deep and from ½ acre surface to 25 acres. Usually the first lagoon is smaller and between 6 and 10 feet deep, while the second lagoon is larger and deeper for storage purposes.

In operation, the animals 718 are processed, for example milked or fed, in the parlor 820, wherein the animals 718 deposit waste material. The parlor cleaning water system may then be used to spray the surfaces of the parlor 820, in particular the parlor floor and any other desired surfaces, with water. The hose 824 may be attached to the nearest convenient hose attachment spigot 822. The water drawn from the parlor cleaning water system includes material drawn from the microorganism-generation tank by way of the feed pipe 870. Therefore, the water used to clean the parlor 820 may include desired microorganisms at a desired rate. The water washes a majority portion of the animal waste material to the lagoon by way of the parlor floor 710 and flush alleys 712. The parlor floor 710 and/or flush alleys 712 may be configured at an incline 930 to create a tendency for fluids to travel from the parlor 820 to the lagoon 827 by gravity. There may be one or more intermediate areas 826 that may include an area of land or poured concrete 826 that may also be at an incline 930, which incline 930 may or may not be to the same degree as the incline 930 of the parlor floor 710 and/or flush alleys 712. The intermediate areas may further include guiding means 28 such as small concrete lips 828 or raised portions 828 configured to guide fluids towards the lagoon 827. The lagoon 827 may typically comprise a barrier 825 containing the contents of the lagoon 823.

The lagoon 827 may be primarily aerobic, anaerobic, or a combination. Further, the lagoon 827 may be a system of lagoons 827 that may be configured in parallel or in series or in some combination. Further, the lagoon 827 may include any number of devices configured to enhance the operation of the lagoon, including but not limited to aerators, circulators, floating cultures, inlets, outlets, covers, and heaters.

While portions of the animal waste material may remain as coatings on surfaces of the parlor 820, in particular the parlor floor 710, the microorganisms in the water may process the remaining animal waste material and any slime present. Processed animal waste material and processed slime are broken into simpler materials which are typically substantially less likely to adhere to parlor surfaces and is likely to be removed to the lagoon 827 on subsequent washings.

Additionally, as desired microorganisms are carried by the water through the parlor 820 to the lagoon 827, the lagoon 827 is populated with desirable bacteria that may increase the rate at which material in the lagoon 827 is processed. Advantageously, this may allow for faster processing of animal waste material. Further, as processed material may be drawn from the lagoon 827 and distributed to plants, the processed material may contain the desirable microorganisms that may further enhance the growth of vegetation.

The microorganism-generation tank may comprise a container 800 configured to contain liquids, an inlet 810 configured to supply water to the container, a feed pipe 870 configured to permit extraction of bio-liquid material from the container 800, and growth and maintenance apparatus (not shown) configured to promote and/or stimulate growth and reproduction of desired microorganisms. Preferably, the feed pipe 870 will be coupled to the container 800 in a lower portion of the container 800. The growth and maintenance apparatus may include but is not limited to aerators, circulators, temperature regulators, nutrients, nutrient regulators, and nutrient disbursement regulators.

In operation of the microorganism-generation tank, microorganisms are generated, or regenerated, as products and further generations of a biological composition, in a liquid mixture in the container 800. Water is introduced into the container 800 by means of an inlet 810 and bio-liquid material is extracted from the container 800 by means of a feed pipe 870. The feed pipe 870 may extract bio-liquid material from the container 800 as needed. The bio-liquid material, or liquid mixture, may be introduced into the feed pipe 870 at a known rate or may be introduced at known portions per elapsed time. Preferably, the rate at which the bio-liquid material is introduced into the feed pipe 870 does not exceed the rate at which the bio-liquid material is generated by the container 800, which is related to the rate at which water is introduced into the container 800 through the inlet 810.

For example, where the container generates, or regenerates, 100 gallons of liquid mixture per day, it is preferable that the rate at which the liquid mixture is introduced, or injected, into the feed pipe does not exceed 100 gallons per day, regardless of whether the rate is constant throughout a twenty-four hour period. One skilled in the art would understand that typically in animal production, routine processes are developed with generally predictable volume usage of cleaning fluids used in production areas.

In operation, the extracted bio-liquid material may mix with the cleaning water at the junction 840. The mixture ratio may be controlled by controlling the relative feed rates of the cleaning water and the extracted bio-liquid material into the junction 840. The combined cleaning fluid produced at the junction 840 may then pass through pipes 860, which may also travel underground 865. The pipes 860 and 865 may so fluidly couple the junction 840 to one or more hose attachment spigots 822, or regulators. One or more hoses 824 may be removably attached to one or more hose attachment spigots 822, thereby fluidly coupling the hose 824 to the junction 840. The hose 824 may then be used to disperse the combined cleaning fluid over surfaces of the parlor 820.

The microorganism-generation tank may beneficially provide a regular supply of desired microorganisms to the parlor cleaning system. In light of this, it is of note that, microorganism generation, and/or regeneration, consumes nutrients. Further, generations of microorganisms tend to mutate, or change, from strains originally introduced. Therefore, preferably, nutrients and original strains of microorganisms are introduced into the container as needed. For example, biological compositions, preferably in pre-made packets including a nutrient quantity and a quantity of original strain(s), or microorganism quantity, are preferably placed within the container on a regular schedule, such as once a week.

In operation of one embodiment, the biological composition may provide a two stage rich source of food and other nutrients for the quantity of microorganisms. The first stage permits rapid reproduction of the microorganism. This beneficially permits the microorganism to predominate other competing microorganisms and creates a large quantity of microorganisms in a short period of time, which then may be dispersed into an animal production facility and/or lagoon. The second stage facilitates maintenance of the microorganism population within the container as portions are extracted and dispersed. During both stages, important nutrients are supplied by the biological composition to the reproducing microorganism population.

Additionally, in operation of one embodiment, the two stage rich source of food is configured to last for a reproduction period, with the materials providing the first stage being substantially consumed before the expiration of the reproduction period. For example, the biological composition may be configured to last for a week, with the first stage configured to be substantially consumed within 48 hours. In another example, the biological composition may be configured to last 4 days, with the first stage configured to be substantially consumed within 24 hours.

One skilled in the art would know that by varying the proportions and total amounts of the materials comprising the ingredients of the two stages and the initial microorganism content of the biological composition, one may adjust the biological composition to generally conform the reproduction period to any reasonable desired period of time. Also, it is not necessary that the reproduction period be known to the manufacturer or that it be designated at all, merely that there be a period of time to which the composition relates as described herein.

Also, in operation of an embodiment, oxygen is introduced to a liquid mixture containing microorganisms in a container. This may be accomplished by injection, bubbling, interface exchange, or any other method known in the art for providing access to oxygen within a liquid. The source of oxygen may be normal air.

Still yet, in operation of one embodiment, portions of a liquid mixture containing microorganisms in a container are extracted from the container. This may be accomplished with an outlet, preferably coupled to a lower portion of the container to avoid clogging with any surface skins formed on a top surface of the liquid mixture in the container.

In addition, the nutrient quantity preferably includes a food source easily utilized by the microorganisms, or short-term nutrient, to promote rapid generation of microorganisms, in particular rapid generation of the original strain. Additionally, the nutrient quantity preferably includes a long lasting food source, or long-term nutrient, configured to nourish the microorganisms after the short-term food source depletes. Also, the nutrient quantity preferably includes a quantity of other nutrients, or supplement nutrients. The nutrient quantity may include but is not limited to vitamins, minerals, enzymes, amino acids, protein compositions, starches, fibers, carbohydrates, sugars, growth media, proteins, chelating agents, complexing agents, sequestering agents, and other materials useful in nourishing microorganisms and plants.

In particular, it is preferred that the microorganism quantity includes a microorganism(s) characterized by the ability to process animal waste, preferably in an animal production facility and/or lagoon. Examples of beneficial microorganisms include but are not limited to bacteria, yeasts, protozoa, actinomycites, and nematodes. It is preferred that the microorganism quantity includes a microorganism(s) characterized by an ability selected from the group consisting of an ability to process nitrogen containing compounds, process phosphorous containing compounds, remove disease-causing organisms from water, facilitate delivery of oxygen to water, convert solid waste to liquid, and benefit any organism characterized by an ability to process nitrogen containing compounds, process phosphorous containing compounds, remove disease-causing organisms from water, facilitate delivery of oxygen to water, and convert solid waste to liquid. Additionally, the microorganisms may be aerobic bacteria. The microorganism(s) may include, but is not limited to bacillus subtilis, bacillus lichenformis, bacillus cereus, bacillus megaterium, fluorescent pseudomonas, azobacter, cellulase enzyme producing bacteria, yeasts, sub-cultures thereof, and combinations thereof.

Still further, it is preferred that the microorganism(s) in the microorganism quantity be included in sufficient quantities to predominate other microorganisms which may use the biological composition to reproduce. “Other microorganisms” as used in the previous sentence may include microorganisms present in a container, in a water supply feeding into a container, strains of similar microorganisms which may have mutated from an original strain related to or identical to the microorganisms present in the microorganism quantity.

Still, in particular, it is preferred that the short-term nutrient include ingredient(s) characterized by the ability to provide a quick and ready source of nourishment for the microorganisms of the microorganism quantity. Preferably, this may include but is not limited to hydrolyzed collagen, bone meal, blood meal, carbon skeleton molecules, sugars, carbohydrates, folvic acid, organic acid, soy protein, peptone treated biological matter (such as peptone treated animal carcasses or peptone treated plant matter), other easily consumed materials and combinations thereof. Preferably, the short-term nutrient is present in the biological composition in sufficient amounts to provide for rapid reproduction of the microorganism quantity and its further generations for a reproduction period.

Further, in particular, it is preferred that the long-term nutrient include ingredients(s) characterized by the ability to provide a stable, lasting (as compared to the short-term nutrient and/or the intended period of replacement of the biological composition packets) source of nourishment for the microorganisms of the microorganism quantity. Preferably, this may include but is not limited to wheat starch, soy flour, molasses, processed or raw animal and/or plant matter, other slowly consumed proteins, fibers, starches, fats and carbohydrates and combinations thereof. Preferably, the long-term nutrient is present in the biological composition in sufficient amounts to provide for continued reproduction of the microorganism quantity and its further generations for a reproduction period after the short term nutrient quantity is substantially consumed.

Again, in particular, it is preferred that the supplement nutrient include ingredient(s) characterized by the ability to provide for the variety of nourishment needs of the microorganisms of the microorganism quantity. It is preferred that the supplement nutrient at least provide for at least one of the non-energy source needs of the microorganisms of the microorganism quantity. Preferably, the ingredient(s) of the supplement nutrient may include but is not limited to food grade proteins; vitamins; inorganic salts; amino acids; growth media; minerals such as phosphate, potassium, calcium, sulfur, cobalt, copper, iron, magnesium, sodium, manganese, and zinc; humate and/or humic acids; enzymes; chelating, complexing, and/or sequestering agents with or without associated molecules; and combinations thereof. Preferably, the supplement nutrient is present in the biological composition in sufficient amounts to nourish the microorganism quantity and its further generations for a reproduction period.

Preferably, the biological composition is in a dry form wherein the biological composition may be stored for a time with the microorganisms in an inactive state. Preferably, the biological composition is a package. Preferably, the biological composition may be powder, granules or a pressed cake. In addition, the biological composition is preferably configured to aid or induce generation or regeneration of a quantity of microorganisms, preferably in a container or system configured to introduce microorganisms into a plant ecosystem. Also, preferably, the biological composition is configured to introduce or reintroduce an early generation of the microorganisms into the container or system. “Early generation” means that the microorganisms are not substantially mutated from the desired species, strains, and/or characteristics.

### EXAMPLE ONE

There is a dry organism amount, including a selection of bacteria, in particular bacillus bacteria that is produced by the organization known under the mark “SafeWaze” that is located at 7411 N. Tryon Street in Charlette, N.C. 28213.

The Bacillus Package, referred to above, includes, but is not limited to specific strains of bacillus including Bacillus Subtilis, Bacillicheniformis, and Bac Megatherium. The microbes are contained in an inert preparation of a natural absorbent which has no chemical impact, combined with powder bran grain as a food source.

There is a dry microorganism amount that includes base soil bacteria, for example, the product known under the brand name Soil Response™. The product known as Soil Response™, is attributed to SafeWaze at 7411 N. Tryon Street in Charlette, N.C. 28213. The product known as Soil Response™ is a mixture of active hydrocarbon oxidizing, natural single-cell organisms, specifically for use on soil including, but is not limited to Pseudomonas Fluorescent, Azotobacter, as well as Cellulase enzymes producing bacteria. The microorganisms are contained in an inert preparation of a natural absorbent that has no chemical impact.

There is a dry nutrient amount which includes a micro-nutrient supplement, a long-term food source and a short term food source. The micro-nutrient supplement is manufactured by the organization having the trademark SafeWaze™ at 7411 N. Tryon Street in Charlette, N.C. 28213. It includes a blend of food grade proteins, vitamins, inorganic salts, and growth media—intended as a supplementary food supply for microorganisms. The micro-nutrient package may include, but is not limited to minerals and nutrients including phosphate, potassium, calcium, sulfur, cobalt, copper, iron, magnesium, and zinc, as well as proteins. The long-term food source is produced under the label DRI-MOL®, which is a dry molasses product manufactured by the organization having the trademark ADM found in Stanley, Wis. 54768. The ingredients include molasses, wheat Starch, calcium strearate, soy flour and lecithin. The short-term food source is hydrolyzed collagen, or HC, of type GCP-1000 which is manufactured by the organization known as Nitta Gelatin NA, INC. at 201 W. Passaic St. in Rochelle Park, N.J. 07662.

The composition amounts are detailed below. There will typically be a period of usage of seven days and a fresh clean out at the beginning of each season.

Biological Package Composition and Application Usage:

| | | Applied into Container on |
|---|---|---|
| | Density | Days 1, 7, 14, 21 |
| Bacillus Package | 3.5 ounces/cup | .5 cups |
| Soil Response TM | 6.25 ounces/cup | 0.625 cups |
| Micro-Nutrient Supple- | 6 ounces/cup | 0.125 cups |
| ment | | |
| DRI-MOL ® | 5.5 ounces/cup | 2.5 cups |
| Hydrolyzed Collagen HC | 3.25 ounces/cup | 0.75 cups |

### EXAMPLE TWO

| | Approximate Percent by Volume |
|---|---|
| Dry Bacteria Culture | 5% |
| Yeast | 1% |
| Soybean Protein | 18.75% |
| Nutrient Mineral Mix | 18.75% |
| Humate | 18.75% |
| Granulated Sugar | 18.75% |
| Flour | 18.75% |

The nutrient mineral mix included crude protein, crude fat, salt, calcium, chlorine, magnesium, phosphorous, potassium, sodium, sulfur, cobalt, copper, iodine, iron, manganese, and zinc.

### EXAMPLE THREE

Examples of materials which are includable in embodiments include a variety of different biological and food mixes including soy protein use for animal feed supplements, mineral mixes used in animal nutrition, protein packages used in body building and dietary supplement, flours, sugar, raw molasses, yeast, various enzymes used in waste treatment, laundry soaps and the product sold under the mark Oxy Clean. Also included are various combinations of products including the product sold under the marks ViBasic, Xcite and ViPlex from the organization under the mark Vitech Industries. Further examples include the product under the mark Esp333, both liquid and dry, as well as a protein feed supplement from the organization under the mark Bio-Kinetics, soil stimulant from the organization known as Fertile Earth, along with various combinations of fertilizers.

### EXAMPLE FOUR

With a three thousand head dairy, having concrete alleys, which are flushed 3 times a day with recycled water out of a second lagoon, the milking parlor is hose washed with fresh water and the cows go through a chemical foot bath prior to milking which is also washed into the lagoon. All flush water goes to a sand separator, then to a transfer basin, pumped to manure separators and then water and smaller solids drain into the first lagoon. It takes approximately 2 to 4 weeks for the waste water to process through the first lagoon. The first lagoon is intended to digest solids. Next the water flows over a weir and into a holding lagoon. In the holding lagoon, the water is used for re-flush water and stored for up to 6 months before being pumped out and dispersed over plants through several irrigation pivots.

A microorganism-generation tank was set up in the utility room of the milking parlor, where fresh water is metered into the tank at a rate of 10 gallons per hour, with an overflow hose leading to a drain that flows to the lagoon. A biological composition with a primary nutrient ingredient produced under the trademark “Bactifeed”, is added to the biological tank two times a week.

A crust had formed over the surface of the first lagoon cover approximately 75% of the surface and between 8 to 24 inches thick. After 30 days open surface area over the lagoon had increased to 50%, and within 3 months 80% of the lagoon surface area was open. After treated water flowed into the storage lagoon the dairyman noticed three things. First, water used in re-flush of alleys left very little slime on the concrete making it safer for cows. Second, pivot nozzle clogging was greatly reduced. Third, odor was greatly reduced, especially as noticed by neighbors during pumping through the pivots.

### EXAMPLE FIVE

There is a six hundred head dairy having a similar layout to that described in Example Four. The dairy is located within a half mile of town and the odor has been an issue for years. A microorganism-generation tank was started in mid summer with the goal of reducing the odor when they drained the lagoons in the fall to make room for winter storage. Later, after being treated by the issue from the microorganism-generation tank for several months, the lagoon was drained and used as irrigation water. Draining the lagoon and using the liquid as irrigation water typically causes substantial odor that triggers many complaints. In this occasion, there was a noticeable reduction in odor and fewer complaints from the town.

### EXAMPLE SIX

here is a farm with 100 milking cows, and 5,000 hogs. The dairy waste is scraped into a holding pit and then shuttled to a lagoon. Waste from the hog pens goes into pits under each hog pen. These pits are drained every two to three weeks into the same lagoon as the dairy waste. At least two problems were addressed by use of a microorganism-generation tank. First, there was a 12 to 24 inch crust covering the entire surface of the lagoon. Second, waste in the hog pits was too viscous to flow easily to the lagoon.

A liquid mixture containing microorganisms was applied to both the hog pits and the dairy holding well on a daily basis. Using several 5-gallon microorganism-generation tanks, a Lagoon composition was mixed with water and let ferment for 24 hours prior to pouring it into the different pits. Regarding the hog pits, application began right after the pits had been emptied and were starting to fill again. When it was time to drain the pits again (3 weeks later) the waste had liquefied to the point that draining them was easier with less scraping and less additional water. The system has continued to improve since. The dairy waste is treated before it is transferred to the waste lagoon. As a result of both treatments the crust on the lagoon is almost completely gone.

It is understood that the above-described preferred embodiments are only illustrative of the application of the principles of the present invention. The present invention may be embodied in other specific forms without departing from its spirit or essential characteristics. The described embodiment is to be considered in all respects only as illustrative and not restrictive. The scope of the invention is, therefore, indicated by the appended claim rather than by the foregoing description. All changes which come within the meaning and range of equivalency of the claims are to be embraced within their scope.

Also, although the figures illustrate a particular cleaning system and tank, it is understood that not all details of the configuration and/or conjunction are shown. Also, it is envisioned that the invention may be adapted to couple with any system for dispersing microorganisms into an animal production facility or animal waste lagoon, or that the invention may be an integral part of such system.

Additionally, although the figure and examples illustrate specific compositions, it is understood that the scope of the invention is to be determined by the claims and not by the specific examples.

It is also envisioned that one embodiment may include one material as a short-term nutrient while another may include that same material as a long-term nutrient, depending on the characteristics of the microorganisms in the microorganism quantity and/or the intended period of packet replacement.

One skilled in the art would know that although the invention is sometimes expressed as a mixture of quantities, it is not necessary that the quantities be distinct. For example, the same ingredient forming the short-term nutrient quantity may also supply at least some of the supplement nutrient quantity, as may be the case where a short-term nutrient quantity includes hydrolyzed collagen.

It is expected that there could be numerous variations of the design and/or configuration of embodiments of this invention. An example is that the biological composition may include additional materials beyond those disclosed. These materials may be carrier materials, inert materials, granulation materials, caking materials, or may or may not serve another function such as a beneficial function related to the packaging, shipping, storing, manufacturing, usage, and/or compliance with authority needs.

It is understood that the above-described preferred embodiments are only illustrative of the application of the principles of the present invention. The present invention may be embodied in other specific forms without departing from its spirit or essential characteristics. The described embodiment is to be considered in all respects only as illustrative and not restrictive. The scope of the invention is, therefore, indicated by the appended claim rather than by the foregoing description. All changes that come within the meaning and range of equivalency of the claims are to be embraced within their scope.

For example, although the illustrations describe particular devices used within the invention, it is understood that any devices performing the same or substantially the same function are to be included as within the scope of the invention. For example, the illustrated power cord may be replaced by RF power transmission. In another example, the illustrated inlet float valve may be replaced by a valve configured to open/close according to a weight threshold.

Additionally, although the figures illustrate particular shapes for tanks/hollow vessels and/or other portions of the invention, it is understood that any suitable shape of these portions may be used. For example, tanks may be rectangular, polygonal, and/or irregularly shaped. In another example, the external shape of a hollow vessel may not correlate with the internal cavity defined thereby. In a still another example, while the pipes and tubes illustrated are generally cylindrical, they may be of any appropriate shape, including but not limited to flat, expandable, rectangular, irregular, polygonal, helical, etc.

It is also envisioned that portions of the regeneration system may be coupled in any known way. There may be joints, welds, solders, screws, rivets, single molded pieces, friction fits, etc. Also, while the regeneration system is illustrated in connection with animal production facilities and with plant irrigation systems, it is understood that the invention includes embodiments for use with any need for a regenerating supply of microorganisms, including but not limited to: pit latrines, septic tank systems, holding tanks, wastewater treatment plants, grease traps, food processing, pretreatment, intensive livestock production, aquaculture, surface water remediation, closed aquatic horticultural or agricultural systems, recirculating systems, remediating waste, extreme environments such as extra-planetary environments, cleaning-in-place, biogas generation, etc.

It is expected that there could be numerous variations of the design of this invention. An example is that a lid present in an embodiment may be centrally located on a top of a hollow vessel, or may be asymmetrically disposed thereon, even on an upper side of the hollow vessel. The bottom of a hollow vessel in one embodiment may be flat, slanted, conical, inverted conical, etc. In another example, while illustrations of the circulation system show the circulation system pulling liquid from proximate the bottom of the hollow vessel and dispersing the liquid into the air above the liquid in the hollow vessel, the circulation system may pull from any portion of the liquid and may disperse to any portion of the system, so as to stir the contents thereof in a manner sufficient to promote reproduction of microorganisms.

Finally, it is envisioned that the components of the device may be constructed of a variety of materials. Preferably the materials are constructed to be resistant to degradation by other materials to which they may be exposed. Portions of the devices included in the various embodiments of the invention may be constructed of fibers, minerals, plastics, metals, composites, ceramics, organic materials, etc.

Thus, while the present invention has been fully described above with particularity and detail in connection with what is presently deemed to be the most practical and preferred embodiment of the invention, it will be apparent to those of ordinary skill in the art that numerous modifications, including, but not limited to, variations in size, materials, shape, form, function and manner of operation, assembly and use may be made, without departing from the principles and concepts of the invention as set forth in the claims.

1. A microorganism regeneration system for providing a regenerating supply of microorganisms for use external to the system, comprising:
a hollow vessel configured to contain liquids and configured to resist degradation by water and microorganisms;
a circulation system disposed within the hollow vessel and configured to circulate and aerate liquids;
an inlet system coupled to the hollow vessel and configured to permit entry of an aqueous liquid into the hollow vessel only when a liquid volume is below a determined threshold; and
an outlet system coupled to the hollow vessel and configured to controllably permit discharge of liquid from the hollow vessel.

2. The microorganism system of claim 1, further comprising:
a microorganism quantity disposed within the hollow vessel, having at least a first generation of microorganisms;
a nutrient quantity disposed within the hollow vessel, in an amount sufficient to sustain reproduction of the microorganism quantity for at least a reproduction period, and
a water quantity disposed within the hollow vessel, in an amount sufficient to cause the liquid volume to be at the determined threshold.

3. The microorganism regeneration system of claim 1, wherein the circulation system comprises a submersible pump configured to circulate liquid within the hollow vessel, wherein the submersible pump is disposed within the hollow vessel and within the liquid volume.

4. The microorganism regeneration system of claim 1, wherein the circulation system comprises a pump providing operating power to circulate and aerate liquid within the hollow vessel.

5. The microorganism regeneration system of claim 1, wherein the circulation system comprises a venturi aerator configured to inject air containing oxygen into liquid contained within the circulation system.

6. The microorganism regeneration system of claim 4, wherein the circulation system provides pressure for discharge of liquid through the outlet system when the outlet system permits discharge of liquid from the hollow vessel.

7. The microorganism regeneration system of claim 1, wherein the circulation system provides pressure for discharge of liquid through the outlet system when the outlet system permits discharge of liquid from the hollow vessel.

8. The microorganism regeneration system of claim 1, wherein the circulation system comprises an intake screen mechanically coupled to a circulation intake and configured to prevent solid materials within the hollow vessel from entering the circulation system while permitting entry of liquids.

9. The microorganism regeneration system of claim 4, wherein the circulation system further comprises a circulation intake disposed within the hollow vessel, hydrodynamically coupled to the pump, and configured to permit entry of liquids contained within the hollow vessel into the circulation system, wherein the circulation intake is sufficiently adjacent the pump to significantly enhance liquid flow around an outer surface of the pump.

10. The microorganism regeneration system of claim 9, wherein the circulation intake is disposed from about 0 inches to about 18 inches away from the pump.

11. The microorganism regeneration system of claim 2, wherein the circulation system comprises a submersible pump configured to circulate liquid within the hollow vessel, wherein the submersible pump is disposed within the hollow vessel and within the liquid volume.

12. The microorganism regeneration system of claim 11, wherein the submersible pump provides operating power to circulate and aerate the liquid within the hollow vessel.

13. The microorganism regeneration system of claim 12, wherein the circulation system comprises a venturi aerator configured to inject air containing oxygen into liquid contained within the circulation system.

14. The microorganism regeneration system of claim 13, wherein the circulation system provides pressure for discharge of liquid through the outlet system when the outlet system permits discharge of liquid from the hollow vessel.

15. The microorganism regeneration system of claim 14, wherein the circulation system comprises an intake screen mechanically coupled to a circulation intake and configured to prevent solid materials within the hollow vessel from entering the circulation system while permitting entry of liquids.

16. The microorganism regeneration system of claim 15, wherein the circulation intake is vertically disposed from about 0 inches to about 18 inches above the pump.

17. The microorganism regeneration system of claim 2, wherein the circulation system comprises a circulation outlet configured to release fluid within the circulation system into the hollow vessel by releasing the fluid into a region of air over the water quantity and within the hollow vessel.

18. The microorganism regeneration system of claim 2, wherein the circulation system comprises a transport tube hydrodynamically coupled to a pump and a circulation intake, wherein the transport tube is configured to provide a pathway to the outlet system and wherein the transport tube is mechanically coupled to the circulation intake by a brace.

19. A microorganism regeneration system for providing a regenerating supply of microorganisms for use external to the system, consisting of:
a hollow vessel configured to contain liquids and configured to resist degradation by water and microorganisms;
a circulation system disposed within the hollow vessel and configured to circulate and aerate liquids, including a submersible pump;
an inlet system coupled to the hollow vessel and configured to permit entry of an aqueous liquid into the hollow vessel only when a liquid volume is below a determined threshold; and
an outlet system coupled to the hollow vessel and configured to controllably permit discharge of liquid from the hollow vessel.

20. The microorganism regeneration system of claim 19, further consisting of a filtered circulation intake disposed from about 0 to about 18 inches away from the submersible pump, and wherein the submersible pump is configured to provide pressure to circulate and aerate liquids within the hollow vessel and to provide pressure to the outlet system.
